(19) United States
(12) Patent Application Publication
(10) Pub. No.: US 2022/0040468 A1
(43) Pub. Date: Feb. 10, 2022

(54) Tamper Resistant and Medical Connector Device

(71) Applicants: Nicholas C. Kreatsoulas, Poland, OH (US); John J. Pastrick, University Hts, OH (US)

(72) Inventors: Nicholas C. Kreatsoulas, Poland, OH (US); John J. Pastrick, University Hts, OH (US)

(21) Appl. No.: 17/397,907

(22) Filed: Aug. 9, 2021

Related U.S. Application Data
(60) Provisional application No. 63/063,127, filed on Aug. 7, 2020.

(51) Int. Cl. A61M 39/16; A61M 39/10

(52) U.S. Cl. CPC A61M 39/162; A61M 39/1011; A61M 2205/0238; A61M 2039/1033; A61M 2205/0205; A61M 2039/1016

(57) ABSTRACT

The tamper resistant medical connector device 10 includes an inner luer cap 30 for threaded sealing engagement with a standard luer SL or PICC line inlet port 14. The inner luer cap is surrounded and enclosed by an outer cap first half component 52 adjacent the inlet port 14, and an outer cap second half component 60 secured to the outer cap first half component to form a unified outer cap 50, and having an cap stem 70 engaged with the inner luer cap 30, on an end of the inner luer cap opposite the inlet port. The cap stem 70 is partially surrounded and enclosed by the outer cap second half 60. Once the threads 32 of the inner luer cap 30 are in threaded engagement with the inlet port, the tamper resistant device 10 resists removal as the outer cap spins freely when rotated in a reverse direction. An unlocking key 100 is used to disengage the tamper resistant device 10 for authorized access to the inlet port 14.

### CROSS REFERENCES TO RELATED APPLICATIONS

This application claims the benefit of and priority to pending U.S. Patent Application Ser. No. 63/063,127 filed 7 Aug., 2020, the entire contents of which is incorporated herein by reference.

### FIELD OF INVENTION

The application relates to medical connector devices including catheters having a locking mechanism for resisting access to catheter lines or inlet ports, and more specifically to peripherally inserted central catheter (PICC) lines, or any common central line, tubing, fitting or syringe, with locking technology to resist unauthorized access to inlets or injection ports. An improved luer and cap having a dual seal is also provided.

### BACKGROUND

As the number of intravenous (IV) drug abusers in the United States continues to increase, the number of drug addicted patients suffering from serious health concerns is also increasing. Abusers of IV drugs such as opioids like heroin often suffer from serious infections, or may require treatment by IV antibiotics and other medications during recovery from surgeries. Longer-term treatment with IV medications is typically accomplished by placing a peripherally inserted central catheter (PICC) line into the patient's venous system during a hospital stay. Once the PICC line is installed by a health care provider, further repeated injections of necessary medications can be easily and quickly accomplished using the PICC line and without the difficulty of finding a vein. After the patient is sufficiently recovered and discharged from the hospital with the PICC line still installed, such repeated medication injections are typically made by health care providers during less expensive out-patient or home care visits. Unfortunately, providing unsupervised access to the PICC line after discharge enables IV drug abusing patients to inject illegal drugs or other substances using the PICC line, and results in increased risk of illegal drug overdoses and death.

With the onset of the corona virus, or SARS-CoV-2, and rising numbers of patients suffering from COVID-19 requiring hospitalization, the need to ensure the availability of hospital beds has become an increasing priority. Additionally, the risk of virus exposure to patients receiving care in a hospital environment may also be increased. As a result, it is increasingly desirable to discharge patients with PICC lines from the hospital as soon as possible.

In order to enable the discharge of patients with a history of drug abuse from the hospital with a PICC line, an improved PICC line, or other catheter, central line, tubing, or inlet port with locking capabilities to resist unauthorized access to injection or inlet ports is needed. One such device that locks a conventional PICC line port within a lidded box is described in U.S. Pat. No. 8,858,505. However, such a device is awkward and inconvenient for a patient to wear, and still further innovations are needed.

Additionally, the need for locking caps on a variety of medical connectors and equipment used in fluid transfer applications has also become apparent. When prefilled syringes containing a desired amount of medication already loaded within the syringe are provided to a health care facility, but without a needle, it is important that the medication remain contained within the syringe until the health care provider is ready to use the syringe during an injection of the medication. Additionally, as with a PICC line, it is important that the syringe and its contents not be tampered with, and remain secure until it is delivered into the hands of the intended health care provider. There is a need for an improved tamper resistant or locking needle-free prefilled syringe cap.

An additional related need has also been found for an improvement in standard luers and caps. Standard luers and fittings typically provide a female threaded end to be covered prior to and following use with a closed cap having an internal threaded connection for engagement with the female threaded end of the luer. Although disinfecting swabs are typically used on the threads of the luer prior to engagement with a cap or cover, a close inspection of the engagement between standard luer threads with the threads of the cap reveal that while a main seal is provided when a tapered post within the cap is wedged within the luer port or opening, the threaded end of the cap does not sufficiently engage with the standard luer to stop contaminants from entering a gap formed between them. An improved double seal luer and cap are needed to ensure a true double seal is provided.

### SUMMARY

A tamper resistant medical connector device is provided for resisting unauthorized intravenous administration of substances to a patient via a common central line, fitting or tubing, such as a peripherally inserted central catheter (PICC) line into the patient's venous system. The device is configured for use with a catheter line having an inlet port in communication with the patient's venous system, but may also be used with a feeding tube or other central line, fitting, tubing or a needle-free syringe having an access or threaded inlet port.

When continuous use of a PICC line is no longer required, and only intermittent use is needed for medical reasons, it is desired to close or secure the inlet port of the PICC line in a manner to prevent access to the inlet port by non-medical personnel. The present tamper resistant medical connector device is provided to resist access to the PICC line inlet port, and to provide an unlocking key to enable medical personnel quickly to remove the device when needed, for authorized access to the PICC line inlet port.

The tamper resistant medical connector device includes an inner luer cap having inner threads for threaded sealing engagement with a standard luer of a PICC line inlet port. The inner luer cap is surrounded and substantially enclosed by an outer cap first or front half adjacent the standard luer, and an outer cap second or back half. The outer cap back half is secured to the outer cap front half to form a unified outer cap. Additionally, a cap stem is engaged with the inner luer cap, on an end of the inner luer cap opposite the standard luer. The cap stem is partially surrounded and enclosed or captured by the outer cap back half.

To engage the tamper resistant medical connector device on the PICC line inlet port, the medical personal grasps the outer cap and engages the inner luer cap onto the standard luer of the PICC line inlet port. By turning the outer cap, the inner threads of the inner luer cap are threaded into engagement with the external threads of the standard luer, while an inner post in the inner luer cap is also engaged into the PICC line inlet port. As the outer cap is pushed and rotated, the inner luer cap moves into sealing engagement with the outer threads of the PICC line inlet port. Once the inner post of the inner luer cap is fully engaged or wedged within the PICC line inlet port, the inner luer cap resists further rotation, and is seated within the inlet port. Once the inner luer cap is locked or seated in position, further rotation of the outer cap is prevented. Reverse rotation of the outer cap also prevents removal of the inner luer cap. In this locked position, ramped surfaces on the tabs of the outer cap back engage mating ratchet surfaces on the inner luer cap such that the outer cap spins freely on the inner luer cap to resist removal. The slope of the ramped surfaces and ratchet surfaces are shallow and slide over one another during rotation, as opposed to engaging and transferring the reverse rotation of the outer cap onto the inner luer cap. As a result, the tamper resistant medical connector device resists removal.

To remove the tamper resistant medical connector device to access the inlet port, a key or an unlocking key is inserted into and along a curved path through aligned openings in the cap stem and outer cap back half. Insertion of the unlocking key causes the key to rotate and align itself within the inner cap as it is fully inserted, until teeth on each leg of the key engage ramped teeth on the inner luer cap. The legs of the unlocking key also include a tab stop, and are sufficiently flexible to snap the teeth and tab stop past a ramped surface on the cap stem, to secure the teeth on the legs of the unlocking key with teeth on the inner luer cap. In this secured position, the legs of the unlocking key are captured within a groove between a circumferential ring and the teeth surrounding an internal surface of the inner luer cap. Once engaged in the secured position with the inner luer cap, the unlocking key may be rotated, along with the entire outer cap, to remove the inner luer cap from the threads of the PICC line inlet port. Upon removal of the inner luer cap of the tamper resistant medical connector device, a health care provider is able to administer authorized medications to the patient through the PICC or catheter line inlet port.

Following administration of any authorized medications, a replacement tamper resistant device is used again to lock the inlet port to resist unauthorized access to the catheter line. Each tamper resistant device is provided with an unlocking key to permit medical personnel to access the PICC line inlet port or other fluid access port.

A tamper resistant medical connector device has been broadly described so that the more detailed description may be better understood. There are additional aspects of the device that will be described below, including a double seal luer or medical connector and cap, and it is understood that more than one embodiment of each device is provided. The devices are capable of still further implementations in addition to those described. It is understood that the terminology used is provided to describe the device, and should not be regarded as limiting, as those of ordinary skill in the art will readily understand the basis set forth here by which other equivalent structures, methods and systems may be provided which do not depart from the spirit and scope of this tamper resistant catheter device.

### DESCRIPTION OF THE DRAWINGS

FIG. 1A is a cut-away view of the tamper resistant device of the present application engaged with an inlet port of a standard luer, and with the unlocking key engaged prior to removal of the tamper resistant device from the inlet port.

FIG. 1A1 is a left side view of the unlocking key shown in FIG. 1A.

FIG. 1A2 is a right side end view of the tamper resistant device of FIG. 1A.

FIGS. 1B1 to 1B4 are a left end view, cut-away view, left side view and right end view of the unlocking key of FIG. 1A1, respectively.

FIGS. 1C1 to 1C4 are a left end view, cut-away view, right side view and right end view of the outer cap first or front half component of the tamper resistant device, respectively.

FIGS. 1D1 to 1D4 are a left end view, cut-away view, left side view and right end view of the outer cap back component of the tamper resistant device, respectively.

FIGS. 1E1 to 1E4 are a left end view, cut-away view, left side view and right end view of the inner luer cap component of the tamper resistant device, respectively.

FIGS. 1F1 to 1F3 are a left end view, cut-away view, and right end view of the cap stem component of the tamper resistant device, respectively.

FIGS. 2A to 2F are cut-away side views of the configurations of the unlocking key from prior to unlocking of the tamper resistant device through full engagement of the unlocking key with the inner luer cap component, prior to removal of the inner luer cap from the inlet port, and FIG. 2G is a cut-away end view of the unlocking key engaged with the components of the tamper resistant device.

FIG. 3 is a top perspective view of the tamper resistant device secured to an inlet port of a PICC line, with an unlocking key positioned prior to insertion for unlocking of the tamper resistant device.

FIG. 4 is a one-third cut-away view of the tamper resistant device of FIG. 3, with an unlocking key positioned prior to insertion for unlocking of the tamper resistant device.

FIG. 5 is a cut-away view as in FIG. 4, but with the unlocking key partially inserted between the outer cap back and the cap stem of the tamper resistant device.

FIG. 6 is a cut-away view as in FIG. 5, but with the unlocking key still further partially inserted between the outer cap back and with the ends of the unlocking key inserted past the cap stem of the tamper resistant device.

FIG. 7 is a cut-away view as in FIG. 6, but with the unlocking key still further partially inserted between the outer cap back and with the ends of the unlocking key inserted nearly into the groove between the circumferential ring and the teeth surrounding an internal surface of the inner luer cap of the tamper resistant device.

FIG. 8 is a cut-away view as in FIG. 7, but with the unlocking key still further partially inserted past the end of the outer cap back, with the tab stops on the legs of the unlocking key shown flexing prior to snapping past the ramped surface on the cap stem, and with the ends of the unlocking key nearly fully inserted into the groove between the circumferential ring and the teeth surrounding an internal surface of the inner luer cap of the tamper resistant device.

FIG. 9 is a cut-away view as in FIG. 8, but with the unlocking key fully inserted, and showing the tab stops on the legs of the unlocking key shown positioned past the ramped surface on the cap stem, and with the ends of the unlocking key fully inserted into the groove between the circumferential ring, and the teeth on the unlocking key engaged with the teeth surrounding an internal surface of the inner luer cap of the tamper resistant device.

FIG. 10 is front perspective exploded view of the components of the tamper resistant device and the unlocking key, and the standard luer or inlet port for a PICC line.

FIG. 11 is similar to FIG. 10, but a rear perspective exploded view of the components of the tamper resistant device and the unlocking key, and the standard luer or inlet port for a PICC line.

FIG. 12 is a top perspective view of an improved double seal luer and cap of the present application.

FIG. 13 is a cut-away view of the double seal luer and cap of FIG. 12, taken long the line 13-13 of FIG. 12.

FIG. 14 illustrates a left end view, cut-away side view, right side view, right end view, and a top plan view (also showing the plane of the cut-away view) of a prior art standard luer engaged with a schematically illustrated standard cap; a left end view, cut-away side view, right side view, right end view, and a top plan view (also showing the plane of the cut-away view) of a prior art standard luer; and a left end view, cut-away side view, right side view, right end view, and a top plan view (also showing the plane of the cut-away view) of a prior art standard cap.

FIG. 15 illustrates a left end view, cut-away side view, right side view, right end view, and a top plan view (also showing the plane of the cut-away view) of an improved double seal luer engaged with a schematically illustrated improved double seal cap of the present application; a left end view, cut-away side view, right side view, right end view, and a top plan view (also showing the plane of the cut-away view) of the improved double seal luer; and a left end view, cut-away side view, right side view, right end view, and a top plan view (also showing the plane of the cut-away view) of the improved double seal cap of the present application.

FIG. 16 illustrates a cut-away side view of the inner luer cap of the tamper resistant device of the present application.

FIG. 17 illustrates a cut-away side view of the prior art standard luer cap shown in FIG. 14.

FIG. 18 illustrates a cut-away side view of the improved double seal cap of FIG. 15.

FIG. 19 illustrates an exploded and partially schematic view of a tamper resistant medical connector device for use in connection with a needle-free syringe.

FIG. 20 illustrates a partially schematic view of an assembled tamper resistant medical connector device of the type shown in FIG. 19 engaged in tamper resistant position with an inlet port of a needle-free syringe, and with an unlocking key positioned for, but prior to, unlocking the medical connector device.

FIG. 21 illustrates the partially schematic view of the tamper resistant medical connector device of FIG. 20, but with the unlocking key in the unlocking position for removal of the medical connector device.

FIG. 22 illustrates a cut-away, exploded and partially schematic view of the tamper resistant medical connector device of FIG. 19, for use in connection with a needle-free syringe.

FIG. 23 illustrates a cut-away, partially schematic view of the tamper resistant medical connector device of FIG. 20.

FIG. 24 illustrates a cut-away, partially schematic view of the tamper resistant medical connector device of FIG. 21.

### DETAILED DESCRIPTION

A tamper resistant medical connector device 10 is provided to resist unauthorized access to a catheter line 12 or other central line, PICC line, fitting, tubing or a needle-free syringe 12′ having an inlet port 14, 14′ in communication with a patient's venous system. The patient is provided with a catheter, central line, fitting or PICC line 20, for example, by a health care provider in a medical facility.

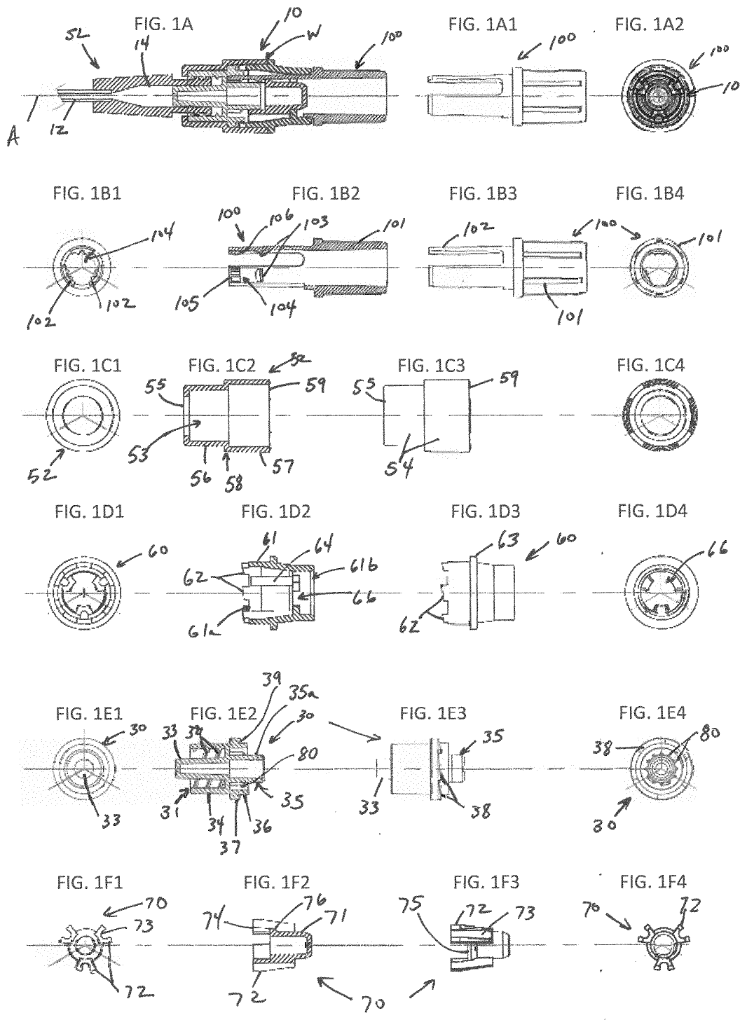

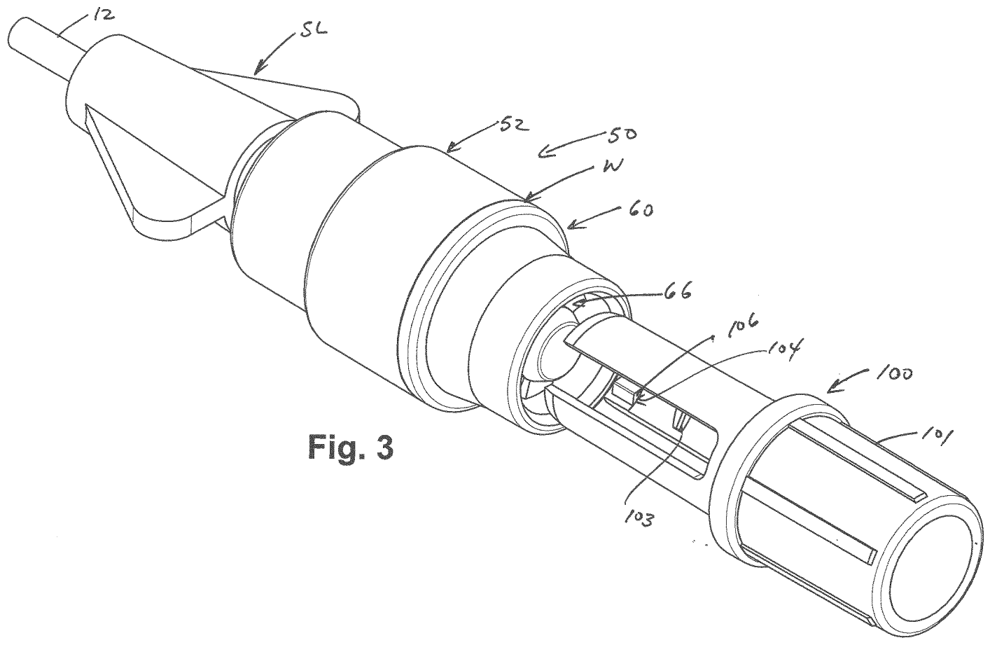

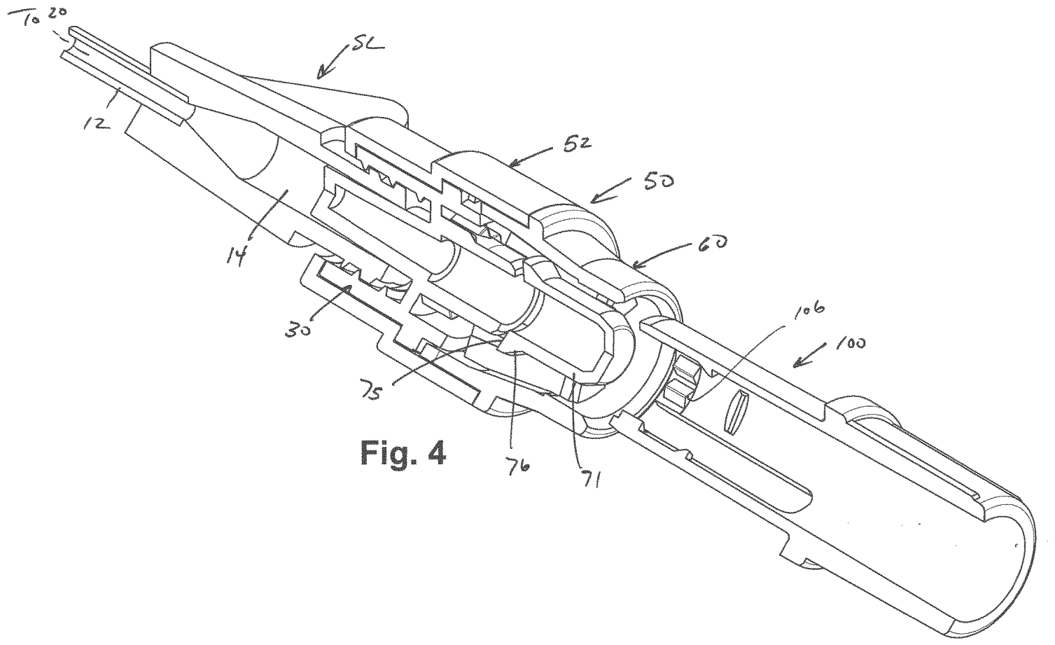

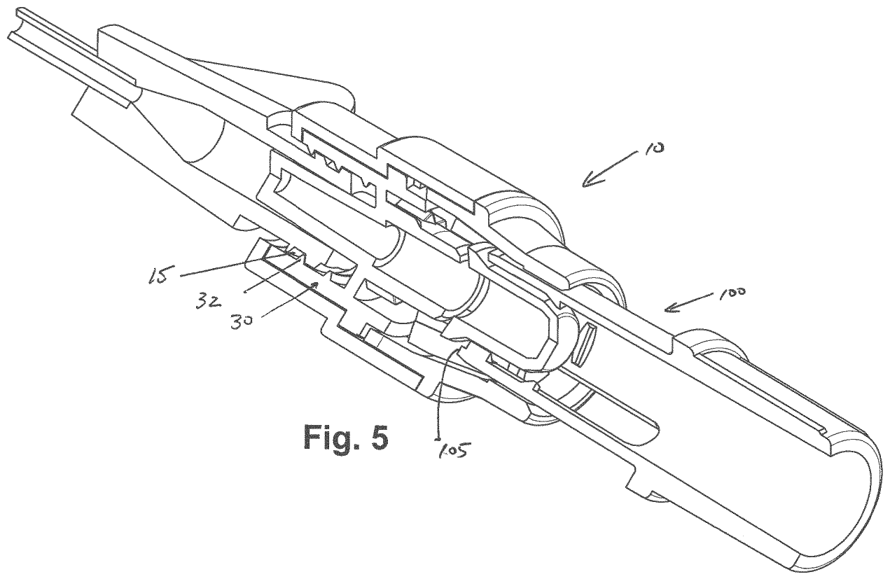

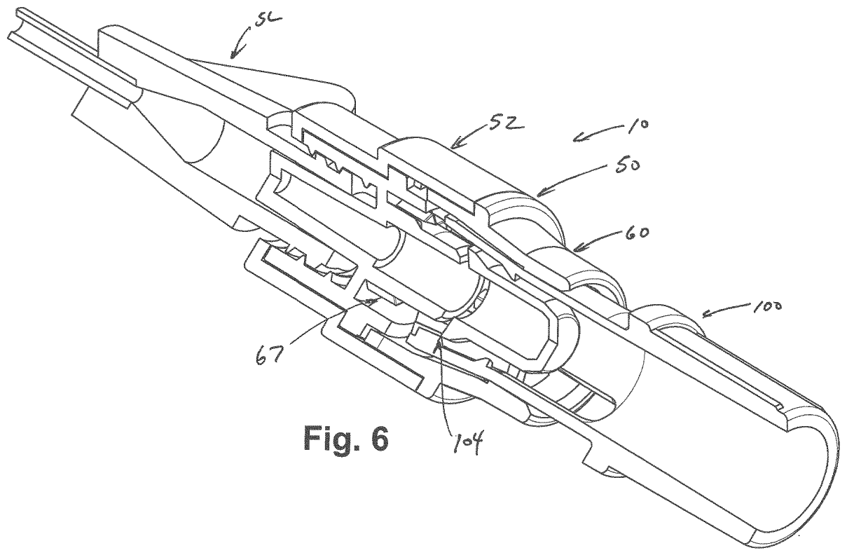

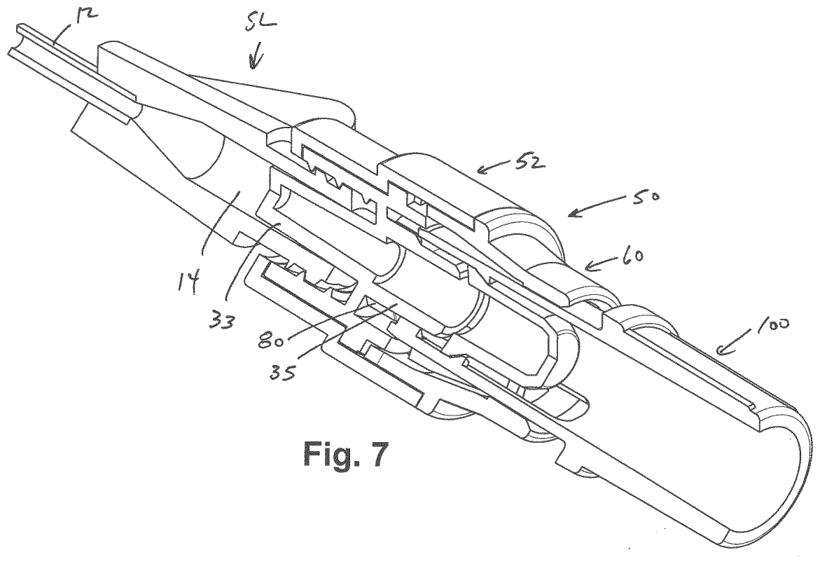

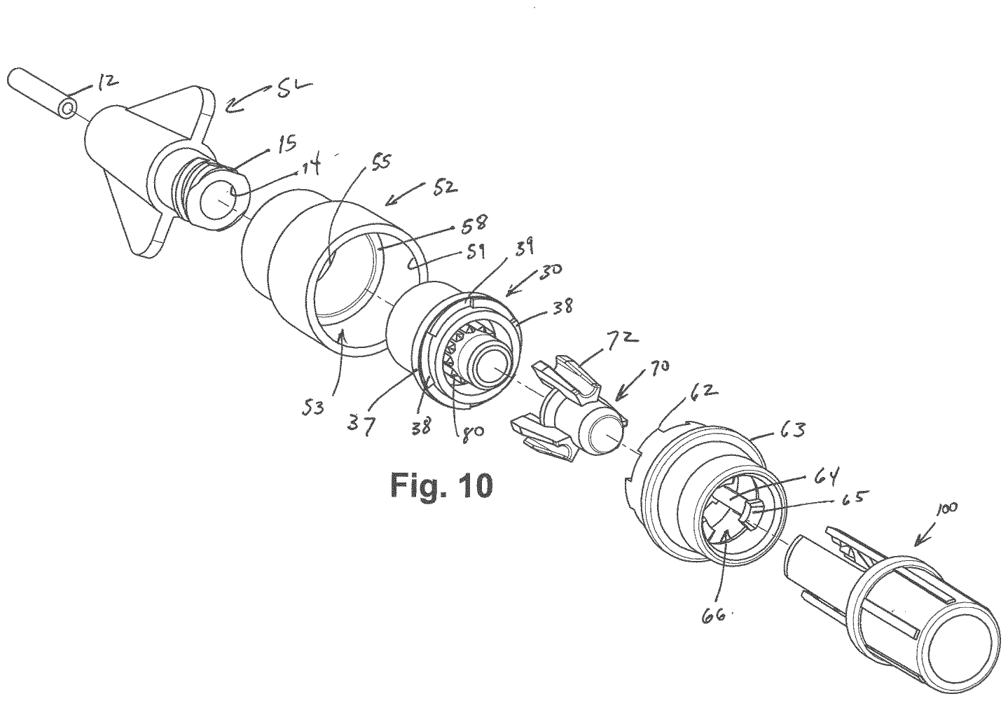

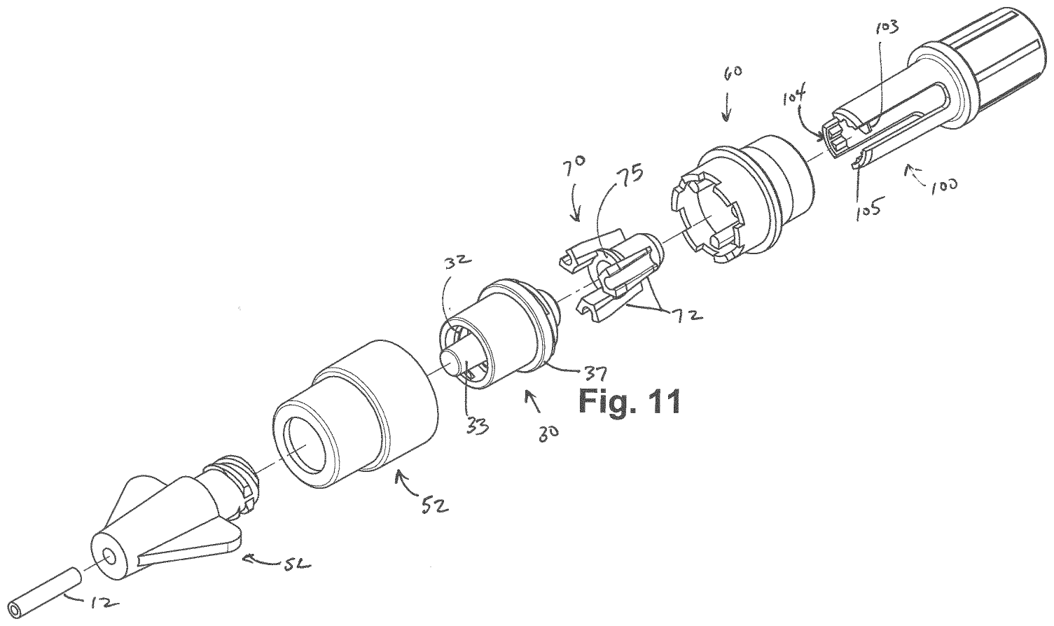

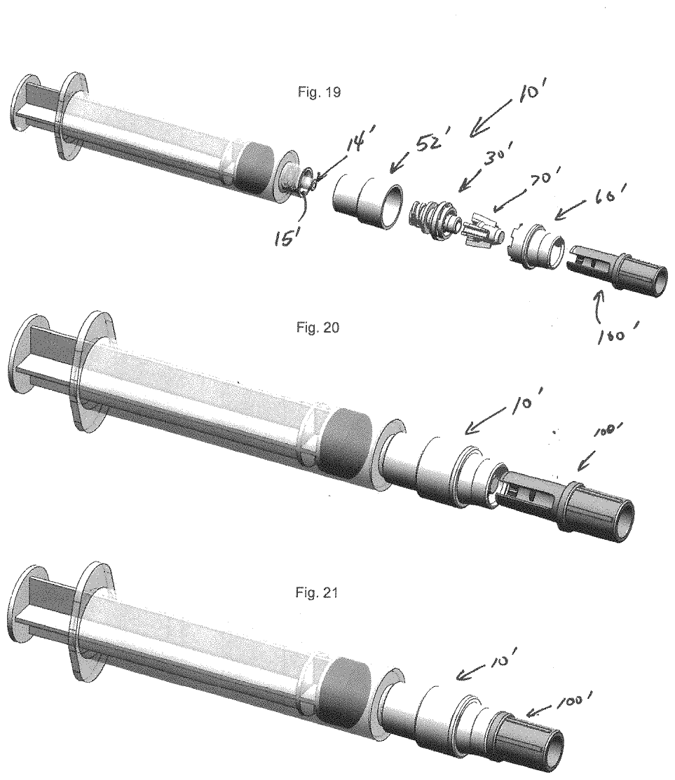

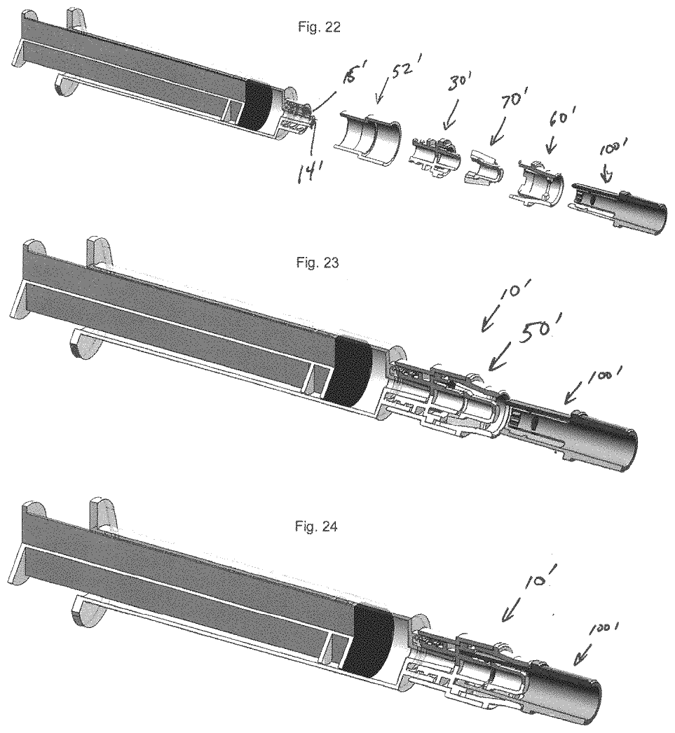

As shown in FIGS. 1A to 11 an embodiment of the medical connector device 10 shown for use in connection with a central line, fitting or other catheter or PICC line is shown. In FIGS. 19 to 24, an alternate, but similar embodiment for use of the tamper resistant medical connector device 10′ with a needle-free syringe 14′ is illustrated. The device 10, 10′ includes an inner luer cap 30, 30′ for threaded sealing engagement with the inlet port 14 or other standard or luer fitting 14′. It should be understood that as the device 10, 10′referenced and illustrated herein may have a variety of similar applications, the use of similar reference numerals, but with a prime designation, may be used to reference similar components in the different applications.

The inner luer cap 30 is surrounded and enclosed by an outer cap first or front half 52 adjacent the standard luer SL, and an outer cap second or back half 60 secured to the outer cap front half 52 to form a unified outer cap 50. The first half and second half components of the outer cap or a locking cap housing 50, are secured together by any appropriate technique, such as adhesives, but the preferred embodiment permanently sonically welds the outer cap first half 52 and the outer cap second half 60 to form the unified outer cap or locking cap housing that surrounds and captures the inner luer cap (while still permitting it to rotate within the outer cap first half 52) and a cap stem 70 component of the tamper resistant device 10 within the locking cap housing. The cap stem 70 is engaged with the inner luer cap 30 on an end 31 of the inner luer cap 30 opposite the inlet port 14 or luer SL. The cap stem 70 is partially surrounded by the outer cap back half 60 and within the outer cap 50. It should be understood that the outer cap back half 60 and cap stem 70 may be formed as a single component or separate components. Likewise, the outer cap front half 52 may be formed as a single component or separate components. Once the components are engaged and the outer cap components are secured together during manufacture, the tamper resistant medical connector device 10, 10′, or locking cap, is presented for use by medical personnel as a single or unified device.

To engage the tamper resistant medical connector device 10 on the PICC line inlet port 14, the medical personal grasps the outer cap 50. The inner threads 32 of the inner luer cap 30 are aligned with the outer threads 15 of the inlet port 14, and pushed and rotated into sealing engagement with the outer threads of the inlet port, which is the same action used to engage a standard luer fitting and cap, and will be familiar to medical personnel. It should be understood that the use of threads on either the inlet port fitting 14 or the inner luer cap 30 may be reversed such that the inner luer cap has external threads, and the fitting has internal threads, for example as shown in the embodiment of FIGS. 19 to 24. As shown in the figures, the components of the tamper resistant medical connector device 10, 10′ are aligned along and rotate around a central axis A.

As shown in FIGS. 1 to 11 and 19 to 24, the locking cap housing 50 defines an open area 53, with the outer cap front half 52 having a cylindrical wall 54 with a first opening 55, a first diameter section 56, a second diameter section 57 that is larger than the first diameter section, a shoulder 58 where the first and second diameter sections meet, and a second open end 59.

The inner luer cap 30 includes a central post 33 surrounded by an internally threaded skirt 34 to removably couple with the externally threaded inlet port 14 through the first opening 55 in the outer cap front half 52. A central protrusion 35 also extends from the inner luer cap 30 in an opposite direction from the central post 33. The central protrusion 35 is surrounded by a skirt 36 having an annular rib 37. The annular rib 37 extends radially outwardly from the skirt 36, and supports ramped ratchet teeth 38 extending from a surface 39 of the annular rib 37 in the opposite direction from the central post 33. An external surface 35a of the central protrusion 35 also supports ramped gear teeth 80, or a repeating array of elements with a unique geometry, within and extending toward an internal surface 36a of the skirt 36.

The outer cap back half 60 is illustrated as a two-piece construction, but may be formed as a single piece. As shown, the outer cap back half 60 includes a circular wall 61 with ramped tabs 62 extending from one open end 61a of the circular wall for mating engagement with the ramped ratchet teeth 38 on the inner luer cap 30. An external annular rib 63 is also provided for sealing engagement with the second open end 59 of the outer cap front half 52, to form the outer cap back half 60 and outer cap front half into a unitary connector device. Internal alignment ribs 64 extend axially inward from the circular wall 61, and internal stops 65 extend inwardly adjacent the alignment ribs 64 and are spaced from a second open end 61b of the outer cap back half circular wall 61. The cap stem 70 is also provided having a cup shaped body 71 with radially spaced ribs 72 extending outwardly from the body. An external surface 73 of the radially spaced ribs is configured for engagement with the alignment ribs 64 of the outer cap back half 60, and a base 74 of each radially spaced rib 72 is positioned adjacent the inner luer cap when the cap stem 70 is engaged within the outer cap back half.

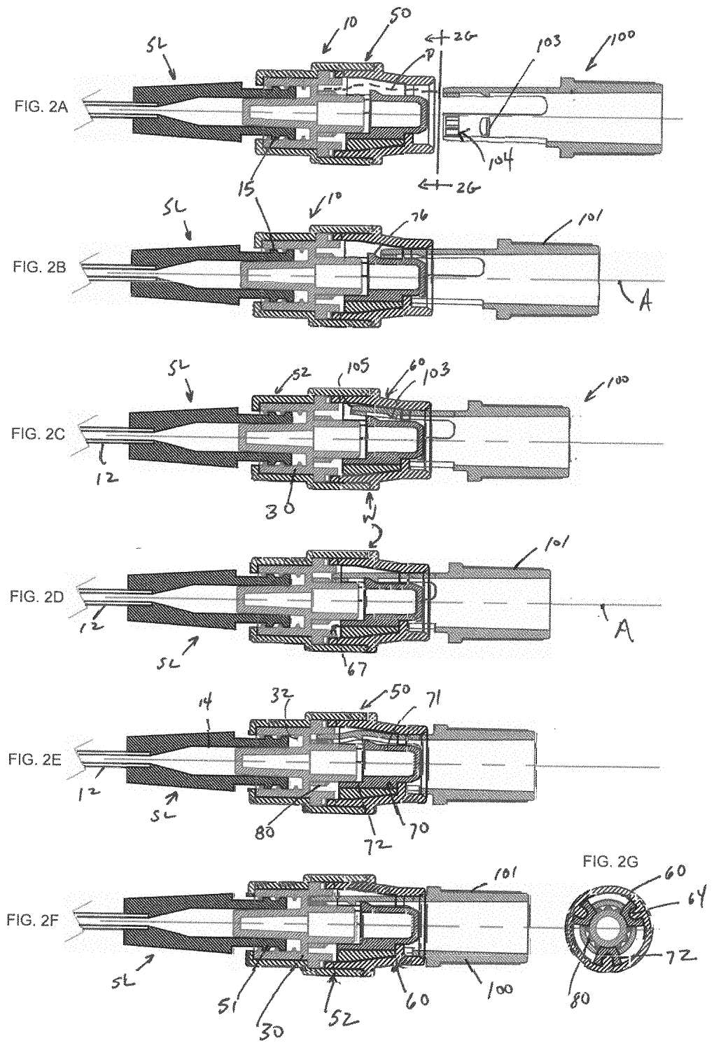

When the medical personal rotates the locking cap housing 50, the inner stem or central post 33 of the inner luer cap 30 rotates to fully engage the inlet port 14 of the standard luer fitting until the inner luer cap 30 resists further rotation, and is seated or sealed within the inlet port. In this seated or locked position, shown in FIGS. 2A, 20 and 23, further rotation of the outer cap 50 is prevented, such that the inner luer cap 30 may not be removed from the inlet port. Likewise, the engagement of the inner luer cap 30 within the locking cap housing 50 on the inlet port 14 places the medical connector 10, 10′ in a tamper resistant resistant position to resist unauthorized access to the patient's venous system via the catheter line 12 and inlet port 14.

Reverse rotation of the outer cap 50 or locking cap is also prevented, such that removal of the inner luer cap 30 is prevented by reverse rotation. When the outer cap 50 is rotated in a reverse direction from the insertion rotation direction, the ramped surfaces on tabs 62 on the outer cap back half 60 engage the mating ratchet surfaces 38 of the inner luer cap 30, but do not operate the ratchet mechanism. While the illustrated geometry of the mating surfaces of the outer cap back half with the inner luer cap are provided as a ratchet mechanism, it should be understood that other surfaces with mating geometry may also be used. In the illustrated embodiment, the mating surfaces are positioned perpendicular to the circumferential direction of rotation of the inner luer cap with the locking cap housing, so that rotation in the clockwise direction tightens the threads of the inner luer cap on the threads of the inlet port fitting until no further rotation is possible. In the case of reverse or counterclockwise rotation of the inner luer cap within the locking cap housing 50, engagement of the ramped surfaces shown in FIGS. 1D3 and 1E3, enable the outer cap 50 to spin freely on the inner luer cap 30 to resist removal. The slope of the ramped surfaces 62 and ratchet surfaces 38 are provided at a sufficiently shallow angle. When combined with smooth mating surfaces and the use of materials with a low coefficient of friction, such as ABS or polycarbonate, for example, rotation by hand or the axial hand force, is insufficient to transfer rotation to the inner luer cap, and the shallow mating ramped and ratcheted surfaces, 62, 38 will slide over one another, as opposed to transferring the reverse rotation of the outer cap onto the inner luer cap. As a result, the tamper resistant medical connector device 10, 10′ resists removal from the inlet port as the locking cap housing spins freely while the inner luer cap 30 is retained on the inlet port. It should be understood that the design could be provided without the ratchet mechanism, such that the outer cap 50 would always spin freely in both directions without the presence of the unlocking key 100. In such a design the unlocking key would be needed for connection of the inner luer cap, and could be removable and reusable.

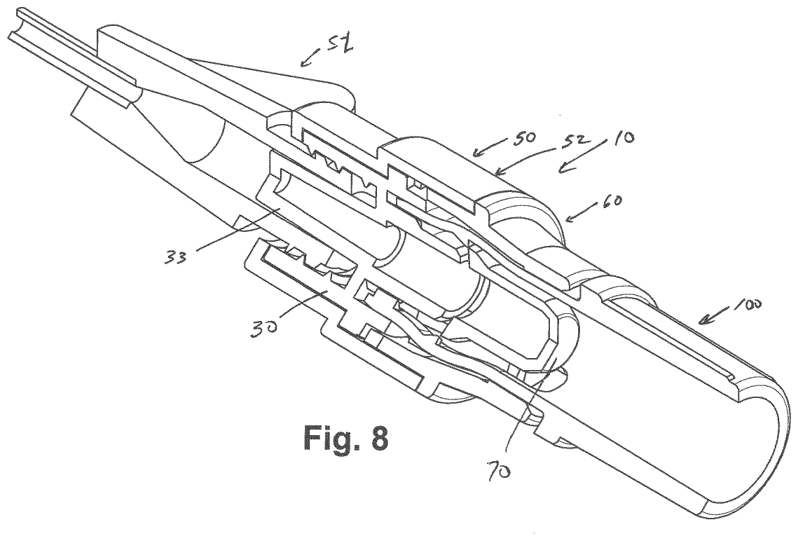

To remove the tamper resistant medical connector device 10, 10′, an unlocking key 100 is inserted into an opening 66 in the outer cap back half 60. The key or unlocking key 100 has a handle portion 101 for rotating the key, and a leg 102. In FIGS. 1B1 to 1B4, at least two spaced legs are shown. Each leg has an internal stop or tab stop 103 extending inwardly from each leg intermediate the ends of the leg. When the spaced legs are inserted into the locking cap housing 50 through a second open end 61b of the outer cap back half 60, the legs 102 are flexed or distorted within the outer cap back half, as shown in FIGS. 2C, 2E and 8, until engaged with a latching surface 75 on the cap stem 70 intermediate the radially spaced ribs 72, and a repeating array of engaging elements, or a pair of teeth 104, on the spaced legs of the key, matingly engage a repeating array of engaging elements, or gear teeth 80, on the inner luer cap. Engagement of the key legs 102 with the inner luer cap 30 permits reverse rotation of the key together with the locking cap housing 50 to an open position and out of engagement with the inlet port.

The ends 105 of the legs 102 are configured for insertion into the openings 66 formed within the outer cap back half of the locking cap housing 50. As shown in FIG. 1B1, the leg 102 is provided with a unique geometry for accessing and engaging the opening 66 also having a unique mating geometry, shown in FIGS. 1D4, 2G and 10 formed within the outer cap back half of the locking cap housing. The legs also include the repeating array of elements, the pair of ramped teeth 104, also having a unique geometry, for mating engagement with the mating gear teeth 80 provided on the inner luer cap 30 to enable coupling of the locking cap housing to the inner luer cap, and permit reverse rotation of the inner luer cap for disengaging the inner luer cap from the threaded inlet port. The leg 102 of the unlocking key 100 is adapted for insertion into the opening 66 along a curved or bent path P formed within the locking cap housing 50. Again, as the legs travel the curved or bent paths through the locking cap, the legs are adapted to bend, flex or distort along the curved path in order to reach the gear teeth 80 on the inner luer cap, matingly engage with the inner luer cap and enable coupling of the locking cap housing to the inner luer cap. Once engaged, reverse rotation of the inner luer cap is enabled to disengage and remove the inner luer cap 30 from the threaded inlet port 14. In the tamper resistant position, the curved path P blocks direct or inline access along the central axis A to the inner luer cap, inlet port 14 and catheter line 12 through the outer cap back half, whether for the purpose of removing the device or in attempt to otherwise access the catheter line 12.

In the illustrated embodiments, the unlocking key 100 has a three-legged, triangular shaped configuration for mating engagement with a triangular or tri-shaped configuration of openings 66 formed in and through the cap stem 70 and the outer cap back half. It should be understood that the use of a single-use unlocking key is preferred in the illustrated embodiments of the figures, since the key is not required for medical personnel to install the tamper resistant device, but only to remove it, thus providing a quick and easy-on device. The unlocking key 100 could be provided with legs having a variety of geometric shapes and configurations for mating with openings having the same geometric shapes and configurations as the locking cap housing. If desired, the key could also be used both for installation and for removal. In addition, once removed with the unlocking key 100, the entire tamper resistant medical connector device together with the key may be readily discarded to an appropriate sharps or other medical hazardous material container.

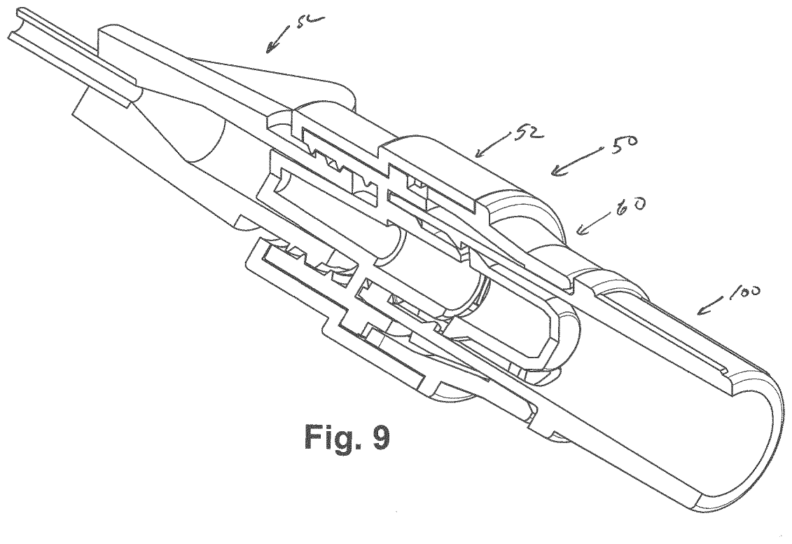

Insertion of the unlocking key 100 through the positions shown in FIGS. 2B to 2F, causes the key to rotate and align itself within the inner luer cap as it is fully inserted, until the pairs or sets of teeth 104 extending inwardly from each leg 102 of the key engage the mating ramped gear teeth 80 formed on the inner luer cap. Each of the three legs of the unlocking key also includes a tab stop 103, and the legs are sufficiently flexible and slidably to enable to the teeth 104 and tab stop 103 to bend past a ramped surface 76 formed on the cap stem 70. The flexibility of the unlocking key legs is important, as the legs must provide the sheer strength necessary to engage and rotate the inner luer cap, while having the necessary elasticity to bend or distort along the curved or blocked path P, including flexing over or past the cap stem 70 and returning to their original configuration. The hooking action provided by the tab stop 103 of the unlocking key legs passing over the ramped surface of the cap stem, to engage a latching surface 75 on the cap stem 70, prevents the key 100 from being removed once it is inserted and latched. However, it is also possible to utilize the back ends 106 of the key teeth to provide a hooking action to retain the unlocking key legs engaged with the inner luer cap. Once the unlocking key is fully inserted into the outer cap 50 as shown in FIGS. 2F and 9, the teeth 104 on the legs 102 of the unlocking key are engaged with the mating teeth 80 on the inner luer cap, and the legs are captured within a groove 67 formed between the skirt 36 and the teeth 80 surrounding the central protrusion 35 of the inner luer cap. Once engaged or coupled within the inner luer cap, the unlocking key may be rotated, along with the entire outer cap 50, also using the ratchet mechanism, to unthread or disengage the threads of the inner luer cap from the threads of the fitting or inlet port. By rotating and pulling the tamper resistant medical connector device and engaged unlocking key along the central axis, both the connector device and the key are removed from the inlet port and discarded. In this unlocked position, authorized medications may be administered to the patient's venous system by medical personnel using the accessible PICC line fitting or inlet port 14 and catheter line 12.

Following administration of any authorized medications, a new tamper resistant medical connector device 10 may be threaded onto the luer threads of the PICC line or inlet port until the device is again locked to resist unauthorized access to the inlet port.

It should be understood that the internal threaded configuration of the inner luer cap 30 may have threads sized and configured for mating interconnection with whichever commercially available fitting is provided in the PICC line fitting inlet port or other fluid access inlet.

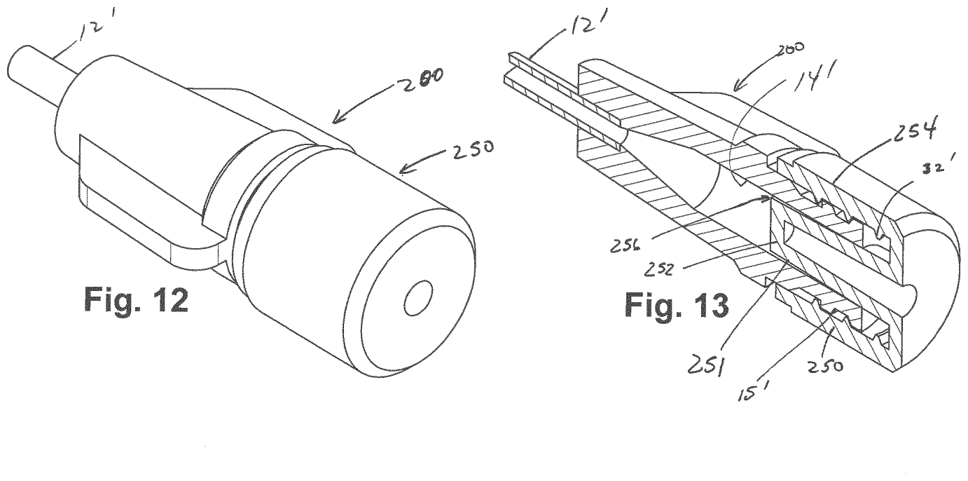

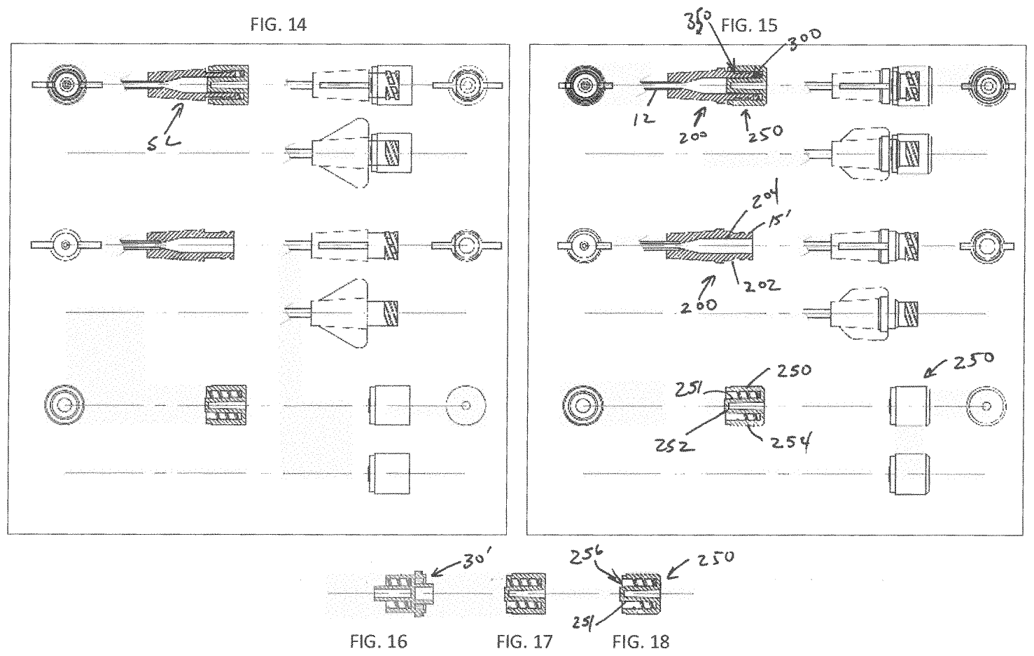

The present application also discloses a new and improved double seal luer 200 and cap 250. As shown in FIGS. 12 and 13, as well as FIG. 15, the improved luer and cap is provided with an internal seal in two locations. Unlike prior art standard luers and caps, shown in FIGS. 14 and 17, which only provide a single seal, the improved double seal luer and cap resists and discourages internal contamination to the threads of the inlet port for intravenous administration of fluid substances to a patient, by providing an additional or secondary seal between the luer and cap. A primary seal 300 in both the prior art and improved luer and cap is provided between an internal inlet port 14′ within the improved luer 200 and a stem surface 251 on the tapered central post 252 within the cap 250, which is surrounded by a threaded skirt 254 to removably couple with the threaded inlet port 14′.

In the improved double seal device, an additional secondary seal 350 is provided between a flared flange or ramped surface 202 adjacent, and extending outwardly from the threaded end of the improved luer in a direction up and away from threads 15′ of the inlet port, to ensure an interference fit between the improved luer 200 and the stem surface 251 internal to the cap 250 adjacent an open end 256. This additional secondary seal 350 does not interfere with the primary seal 300 between the luer inlet port and the central post 252 of the cap. The improved double seal device has a slightly longer length at the base of the luer, as compared with a prior art standard luer, shown in a comparison of FIGS. 14 and 15. At the base 204 of the improved double seal luer, a gap is always provided so that the primary seal 300 is able fully to engage prior to any engagement or interference fit in the secondary seal. As the threads are tightened down, the primary seal 300 tightens with a higher radial compression, while the secondary seal 350 has a lower radial compression. No outer or secondary seal is provided in prior art devices, meaning contamination may more easily enter the threaded portion of the standard luer and cap. The improved double seal luer 200 and cap 250 provide an additional secondary seal 350 to enable increased resistance to contamination of the threaded portions of the double seal luer and cap. In the improved double seal luer and cap, the entire threaded portion 15′ of the inlet port 14′ fitting is isolated or closed off from exposure to atmosphere or the environment.

The various components of the tamper resistant device 10, 10′ and double seal luer 200 and cap 250 may be made from any suitable medical material. Examples of suitable materials include plastics or other polymers, resins, metals or the like. In particular, the locking components may be made from a relatively hard plastic such as ABS or polycarbonate, as well as other thermoplastics. The components may be formed in any suitable manner. Examples of suitable forming methods may include, for example, injection molding, molding, 3D printing, milling, or the like.

It should be understood that any desired color-coding of the locking caps (for example, red), locking sleeves (for example, blue) and removal tools (for example, gold or green) to accomplish ease of use may be included with the tamper resistant devices disclosed here. In addition to the above mentioned tamper resistant or PICC line lock out devices, additional benefits to include with lock out devices include providing an anti-microbial layer or coating of material on an inside surface or surfaces of the inner luer cap, in order to treat the threaded end of the lumen adapter component adjacent the inlet port of the PICC line for reducing infection. In the case of the double seal luer and cap, an anti-microbial agent may also be provided, and could be captured within a sealed area located between the primary and secondary seals to provide greater longevity to the effective life of the agent

It should be further understood that the tamper resistant device is not limited to the particular construction and arrangement of parts illustrated and described here, but embraces all such modified forms as come within the scope of the following claims.

We claim:

1. A tamper resistant medical connector for resisting unauthorized intravenous administration of substances to a patient comprising,
a catheter line having a threaded inlet port;
an inner luer cap having a threaded wall for removably coupling with the threaded inlet port; and
a locking cap housing having an outer cap first half and an outer cap second half, wherein the inner luer cap is substantially surrounded within the locking cap housing, and
wherein when the inner luer cap and threaded inlet port are coupled together in a tamper resistant position, the medical connector resists removal of the locking cap housing and inner luer cap from the threaded inlet port.

2. The tamper resistant medical connector of claim 1, wherein the outer cap first half and the outer cap second half are permanently secured together and rotatably capture the inner luer cap within the locking cap housing to enable the inner luer cap to spin freely within the outer cap first half upon rotation of the outer cap second half in the tamper resistant position.

3. The tamper resistant medical connector of claim 2, wherein the outer cap first and second halves are comprised of one or more components.

4. The tamper resistant medical connector of claim 2, further comprising an unlocking key having a leg for insertion into an opening having a unique geometry formed within the locking cap housing, the leg having a mating unique geometry for accessing and engaging the unique geometry opening formed within the locking cap housing, the leg further including an element also having a unique geometry, for mating engagement with a repeating array of elements with mating unique geometry provided on the inner luer cap to enable coupling of the locking cap housing to the inner luer cap, and causing reverse rotation of the inner luer cap to disengage the inner luer cap from the threaded inlet port.

5. The tamper resistant medical connector of claim 2, further comprising an unlocking key having a leg for insertion into an opening to a curved path formed within the locking cap housing, the leg adapted for accessing the curved path formed within the locking cap housing by bending or distorting along the curved path to reach the inner luer cap, the leg further including an element having a unique geometry, for mating engagement with a repeating array of elements with mating unique geometry provided on the inner luer cap to enable coupling of the locking cap housing to the inner luer cap, and causing reverse rotation of the inner luer cap to disengage and remove the inner luer cap from the threaded inlet port.

6. The tamper resistant medical connector of claim 4, wherein the unlocking key has two or more spaced legs for insertion into two or more corresponding spaced unique geometry openings formed within the locking cap housing, and the unique geometry element on each leg comprises a pair of teeth extending from an internal surface of each leg for engagement with the repeating array of elements having a unique geometry comprising mating teeth formed on an internal surface of the inner luer cap.

7. The tamper resistant medical connector of claim 5, wherein the unlocking key has two or more spaced legs for insertion into two or more corresponding spaced openings to access two or more associated curved paths formed within the locking cap housing, the unique geometry element on each leg comprising a repeating array of elements or a pair of teeth extending from an internal surface of each leg for engagement with the repeating array of elements having a unique geometry comprising mating teeth formed on an internal surface of the inner luer cap.

8. The tamper resistant medical connector of claim 7, wherein the outer cap second half includes the opening to the curved path, and the opening has a unique geometry for engagement by the key leg having a mating unique geometry, and wherein upon passing through the opening and along the curved path, the key distorts to reach the inner luer cap where the unique geometries of the repeating array of mating elements are engaged for removing the inner luer cap from the threaded inlet port.

9. The tamper resistant medical connector of claim 5, 7 or 8, wherein the outer cap second half includes a latch along the curved path formed within the locking cap housing, wherein upon insertion and distortion of the key leg past the latch, the key leg engages the latch to resist removal from the locking cap housing.

10. The tamper resistant medical connector of claim 4, wherein the inside surface of the inner luer cap includes a coating of anti-microbial material.

11. A medical connector for controlling the flow of fluid through a threaded inlet port for administration of intravenous fluids to a patient, the medical connector comprising
a locking cap comprising a locking cap housing defining an open area, and having an outer cap front half and an outer cap back half, the outer cap front half having a cylindrical wall with a first opening, a first diameter section, a second diameter section that is larger than the first diameter section, a shoulder where the first and second diameter sections meet, and a second open end,
an inner luer cap having a threaded wall to removably couple with the threaded inlet port through the first opening in the outer cap front half, a central tapered protrusion extending from the inner luer cap on an opposite end from the threaded wall, which central tapered protrusion is surrounded by a skirt having an annular rib extending radially outwardly from the skirt, the annular rib supporting ramped ratchet teeth extending from a surface of the annular rib in a direction toward the opposite end from the threaded wall, and an external surface of the central tapered protrusion supporting ramped gear teeth within and extending toward an internal surface of the skirt,
the outer cap back half comprises a first part having a circular wall with ramped tabs extending from one open end of the circular wall for mating engagement with the ramped ratchet teeth on the inner luer cap, an external annular rib for sealing engagement with the second open end of the outer cap front half, and an inner portion or stem having at least one opening in the outer cap back half and creating limited access to the inner luer cap by forming at least one blocked or curved path through the outer cap back half, stem and inner luer cap to prevent access to the inlet port; and
wherein when the locking cap is in a tamper resistant position, the locking cap housing rotatably captures the inner luer cap engaged within the open area of the outer cap front half, the stem engaged within the first part of the outer cap back half, and by coupling the threaded wall of the inner luer cap with the threaded inlet port, the locking cap resists removal from the inlet port.

12. The medical connector of claim 11, wherein sealing engagement between the external annular rib of the outer cap back half and the second open end of the outer cap front half comprises a weld.

13. The medical connector of claim 12, wherein the inlet port for administration of intravenous fluids is externally threaded, and the inner luer cap has a tapered central post surrounded by an internally threaded skirt for removably coupling the inner luer cap with the externally threaded inlet port.

14. A tamper resistant medical connection system for resisting unauthorized access to an externally threaded inlet port for administration of intravenous fluids to a patient, comprising:
a medical connector that controls the flow of fluid through the externally threaded inlet port, the medical connector comprising
a locking cap comprising a locking cap housing defining an open area, and having an outer cap front half and an outer cap back half, the outer cap front half having a first opening and a second open end,
an inner luer cap having a tapered central post surrounded by an internally threaded skirt to removably couple with the externally threaded inlet port through the first opening in the outer cap front half, and an annular rib supporting ramped geometry and a repeating array of engaging elements;
the outer cap back half having a circular wall with tabs extending from one open end of the circular wall and having ramped geometry for mating engagement with the ramped geometry on the inner luer cap, and a cup shaped body with radially spaced ribs and a latching surface intermediate the radially spaced ribs;
wherein when the locking cap is in a tamper resistant position, the locking cap housing contains the inner luer cap engaged within the open area of the outer cap front half, within the outer cap back half, and the outer cap back half is permanently secured to the second open end of the outer cap front half, and wherein by coupling the internally threaded skirt of the inner luer cap on the externally threaded inlet port, the locking cap resists removal from the inlet port when the inner luer cap spins freely within the locking cap.

15. The tamper resistant medical connection system of claim 14, wherein the locking cap, including the inner luer cap, outer cap front half and outer cap back half, each have a central axis, and the central axes of the locking cap of the medical connector are aligned when in the tamper resistant position.

16. The tamper resistant medical connection system of claim 14, further comprising a key for removal of the locking cap from the externally threaded inlet port to permit the flow of fluid through the inlet port, the key comprising a handle portion for rotating the key, and at least two spaced legs, each leg having an internal stop extending inwardly from each leg, and wherein when the spaced legs are inserted into the locking cap housing through a second open end of the outer cap back half, causing the legs to flex or distort within the outer cap back half until engaged with latching surfaces intermediate the radially spaced ribs, and a repeating array of engaging elements on the spaced legs of the key matingly engage a repeating array of engaging elements on the inner luer cap, engagement of the key with the inner luer cap permits reverse rotation of the key together with the locking cap to an open position out of engagement with the inlet port.

17. The tamper resistant medical connection system of claim 14, wherein the inside surface of the inner luer cap includes a coating of anti-microbial material.

18. The tamper resistant medical connection system of claim 16, wherein the key comprises three spaced legs for flexible engagement through radially spaced ribs of the outer cap back half.

19. The tamper resistant medical connection system of claim 18, wherein the spaced legs of the key are sufficiently flexible to bend outwardly over the outer cap back half and to return inwardly and maintain engagement with the latching surfaces of the outer cap back half.

20. The tamper resistant medical connection system of claim 16, wherein the locking cap and key are color coordinated for indicating proper connections between the inlet port, locking cap and key.

21. A method for resisting the unauthorized intravenous administration of fluid substances to a patient comprising the steps of:
coupling a threaded inlet port used for administration of intravenous fluids to a patient with a mating threaded inner luer cap of a tamper resistant medical connector to resist removal of the tamper resistant medical connector from the threaded inlet port when the inner luer cap is substantially surrounded by a locking cap housing, the locking cap housing together comprising an outer cap front half and an outer cap back half secured together such that ratchet teeth on the inner luer cap slide past mating ratchet teeth tabs on the outer cap back half, whereupon rotating the locking cap housing in either direction spins the locking cap housing but does not disengage the inner luer cap from the threaded inlet port;
inserting an unlocking key into the outer cap back half to align and engage mating teeth on the unlocking key and on the inner luer cap, and maintaining the unlocking key engaged within the tamper resistant medical connector; and
rotating the unlocking key together with the tamper resistant medical connector to disengage the threaded inner luer cap from the threaded inlet port.

22. A medical connector and cap for threaded engagement covering an inlet port for intravenous administration of fluid substances to a patient,
the medical connector comprising a threaded inlet port and having a ramped surface extending outwardly from an external surface of the inlet port that is spaced and ramped in a direction up and away from threads of the inlet port, and
the cap comprising an open end and a tapered central post surrounded by a threaded skirt to removably couple with the threaded inlet port,
wherein a first seal is provided between an internal passage or surface of the inlet port of the medical connector and the tapered central post of the cap, and a second seal is provided between the ramped surface on the external surface of the inlet port and an internal surface of the cap adjacent the open end which second seal encloses the threads of the inlet port and threads of the threaded skirt to isolate the threads from exposure to the atmosphere.
